(19) United States
(12) Patent Application Publication
(10) Pub. No.: US 2021/0369101 A1
(43) Pub. Date: Dec. 2, 2021

(54) Patient Eyesight Exam and Referral System

(71) Applicant: Zaina Ghouri, San Diego, CA (US)

(72) Inventor: Zaina Ghouri, San Diego, CA (US)

(21) Appl. No.: 17/331,483

(22) Filed: May 26, 2021

Related U.S. Application Data
(60) Provisional application No. 63/029,893, filed on May 26, 2020.

(51) Int. Cl. A61B 3/032; G16H 40/67; G16H 50/20

(52) U.S. Cl. CPC A61B 3/032; G16H 50/20; G16H 40/67

(57) ABSTRACT

A device and system providing remote eye examinations at examination stations is provided. Using patient-operated vision testing at the remote examination stations, a vision determination of a user is ascertained. Should eyeglasses be determined to provide vision correction for such a vision determination, the patient or a parent of the patient is provided a communication of such in a message. The patient can also be referred to a local eye care professional for treatment and provision of corrective lenses.

### BACKGROUND OF THE INVENTION

### 1. Field of the Invention

This application claims priority to U.S. Provisional Patent Application Ser. No. 63/029,893, filed on May 26, 2020, which is included herein in its entirety by this reference thereto.

The invention herein disclosed relates generally to eyesight exams. More particularly, it relates to multi-location system for the provision of improved eyesight examinations for patients such as children and concurrent referral of patients in need of vision correction to a local vision professional.

### 2. Prior Art

### BACKGROUND OF THE INVENTION

In the United States and many industrialized nations a majority of early vision examinations for young children are handled by schools which the children attend. Such examinations are generally conducted by a school nurse or medical contractor and are adapted to identify child patients with minor as well as major vision issues. It is thought that the schools started and continue such eye examinations in order to identify and try to help students who have vision issues that might impair reading and/or viewing the chalkboard or visual projections employed by an instructor at a distance from their seating assignment.

While such eye exams generally are successful in identifying vision issues for particular students, they do nothing to actually provide for the correction of the vision of identified students needing such. Conventionally, it is left to the school administration to contact the parents of a student in need of vision correction and simply inform them of the findings of an informal eye examination.

Parents of such children, however, frequently have busy schedules and careers of their own. Consequently, even if notified by a school of a vision issue of their child, the step of actually securing an appointment for an eyesight professional such as an optician or an ophthalmologist to further test the vision of their child goes unfinished.

Such failure to both identify vision issues and provide professional examination followup can result in significant problems to the child. First, they will lack clear vision for reading and viewing of projected or chalkboard lessons. Further, students identified with a vision problem, because they are children, may never have perceived actual visual clarity, because they have no benchmark for comparison. If such vision problems are not corrected by substantially the age of twelve, they will experience vision loss which is permanent and cannot be corrected later in life with eyeglasses. Such a tragic but easily avoided outcome is conventionally known as amblyopia.

Still further, conventional eyesight tests performed on students in schools have not changed much in decades. Such tests generally involve having the student cover an eye and then identify differing sized letters on a chart. For students who are too young to know the alphabet, directional symbols are displayed for the child to identify. Neither type of test is interesting nor can be considered a fun endeavor, and consequently, the interest of the student being tested is at best lacking. Such a lack of interest and resulting lack of attention during the test can easily cause an inaccurate outcome.

With respect to the above, before explaining at least one preferred embodiment of the system herein for examining the eyesight of patients such as children in general, it is to be understood that the eyesight examination and referral system invention is not limited in its application to the details of employment and to the arrangement of the components or the steps set forth in the following description or illustrated in the drawings. The various apparatus and methods and steps of the vision testing and followup system herein are capable of other embodiments, and of being practiced and carried out in various ways, all of which will be obvious to those skilled in the art once the information herein is reviewed.

Also, it is to be understood that the phraseology and terminology employed herein are for the purpose of description and should not be regarded as limiting. As such, those skilled in the art will appreciate that the conception upon which this disclosure is based, may readily be utilized as a basis for other vision testing systems providing followup professional referrals.

It is important, therefore, that the embodiments, objects and claims herein, be regarded as including such equivalent construction and methodology insofar as they do not depart from the spirit and scope of the present invention.

### SUMMARY OF THE INVENTION

The disclosed software and network enabled system herein, and remote located examination sites, provides both a software enabled system and physical locations enabling the eyesight examination of patients, especially children. The system employs an eyesight examination station or stations which are preferably portable or removably installed at venues where users can employ the station for an eyesight examination. The examination stations, in one mode, may be moved to a school or similar venue on a particular date, for the scheduled performance of eyesight examinations. The examination station can also be positioned in venues frequented by adults, parents and children alike, such as malls, airports, pharmacies, and the like for the provision of unscheduled and free eyesight examinations.

Initially, at the eyesight examination station, the patient is preferably presented with a graphic display having input areas thereon. The graphic interface and display will allow the patient to at least input their name and electronic or mail contact information, and preferably a geographic location for the home or work location of the patient. A menu or drop down driven graphic depiction can ask the questions of the user and input the responses to the questions for such information to electronic memory.

The input information is electronically communicated to and held in stored information specifically related to the patient and patient identifier, in a standardized format about a patient's information and location in one or a plurality of network-based non-transitory storage devices such as having electronic memory in communication with the host computer or server. The host computer or server has a computer processor and electronically engaged electronic memory in which software is running to the task of accepting such patient information and storing it in the standardized format in one or a plurality of network-based non-transitory storage devices.

In cases where the examination station is located at a school, such patient contact information and location information may be input by a school employee or test administrator. Where the patient is a minor at a school, all such patient information will then be associated to the parents or guardian of the individual student. The patient identifier will be associated with the student taking the test, but all contact information will correlate only to the parent or guardian contact information such as their email and/or address.

An eyesight examination station will, in a currently preferred mode of the system, provide a physical wheel having a plurality of examination lenses thereon, positioned adjacent the circumference of the wheel. The lenses are similar to those employed by a phoropter used in an eye professional's office.

Once the above noted personal and geographic location information is stored and related to the patient identifier, during the self-administered test, the patient is directed to view a vision exam image or indicia such as alphabet letters or a standard eye chart, operatively positioned on the examination station, through each of a plurality of examination lenses. Viewing the vision examination image or indicia through multiple lenses allows the patient to determine which vision examination image is discerned or seen as clearest or most focused when viewed through an individual one of the plurality of examination lenses, by its positioning next to a specific vision determination image.

Adjacent each of the plurality of examination lenses is positioned a vision determination image, such as an icon or indicia of other graphic depiction such as of an animal. Such icons or indicia for the vision determination image for example, in a preferred mode of the system for children, will be depictions of well-known animals such as a turtle, a tiger, a panda, and similar images which depict animals. Other images recognizable by children and/or adults may be employed for the vision determination images, for example, depiction of a car, fire engine, or palm tree. The object that is adjacent each examination lens, through which the patient views the examination letters or indicia, is a vision determination image which is easily recognized by the patient.

Upon completion of testing of each eye by a patient, a means to communicate the vision determination image located adjacent to the individual examination lens through which the patient viewed the vision examination image clearest or most focused, is provided. Such is currently preferred as a graphic depiction positioned as an icon or area for touch on a video display of the examination station in which the patient will touch or drag the vision determination image. In some cases where the child is too young to understand how to input the vision determination image, the depiction on the graphic display of the examination station may be or include an oral communication to a human or to a computing device using speech recognition.

The patient will employ the lenses of the examination station to examine both eyes and the identifier of respective vision determination image chosen by the patient for each eye of the patient is communicated electronically, as a vision test outcome, over the computer network to the host computing device. The host computing device has electronic memory or non-transitory storage operatively engaged with the computer processor for running software configured to the task of receiving each vision determination image, for each eye tested on the station, and associating it with the patient identifier assigned to that patient.

Software, running in electronic memory in communication with the host computer or server, will determine a vision outcome for each vision determination image, so communicated, for each patient using the station. The vision outcome, such as near-sighted or far-sighted, etc. and patient identifier information held in host computer engaged electronic memory or non-transitory storage which may be local or in communication over a network, such as the internet, is transmitted from the local computer device to a remote server.

In arriving at a vision outcome for each eye of a patient who has employed the station for an exam, the system will employ software or machine executable instructions which will receive the vision test outcome and employ the patient chosen vision determination image for each eye to thereby determine the determined vision outcome of that patient in each eye. Based on the determined vision outcome for each eye of the respective patient and using a database of vision corrections available for each determined vision outcome, the software will operate to the task of a correction determination and if an optical correction is needed for one or both eyes of the patent based on the input from the testing station.

Such a correction determination may be, for example, normal vision, or may be near-sightedness, far-sightedness, astigmatism or other vision issue. Upon any correction determination that physical eyewear is needed or is available for optical correction for the patient, the software running in electronic memory of the host computer or other network-engaged computer will then ascertain a local eye care professional's office which is located proximate to the geographic location associated with the patient. Such a determination will employ location software running in electronic memory which communicates with a stored eye care professional database in electronic memory of eye care professionals, to determine one or more of the eye care professionals which are proximate to the geographic location input by the respective patient. By proximate is meant within one hundred miles of the input geographic location of the patient. In a preferred mode of the system herein, the software, operating to compare the input patient location, will ascertain the geographically closest eye care professional held in the eye care professional database held in non-transitory or electronic memory, as the chosen eye care professional.

If the patient is an adult, the contact information input by the adult patient at the station may be communicated directly to the chosen local eye care professional. This communication of the adult patient contact information allows the chosen eye care professional to directly contact the patient directly to schedule an appointment. If the patient is a minor, the system will communicate to the parent, guardian or to the school, the correction determination or outcome of the vision test and will communicate to the parent the contact information for the chosen local eye care professional determined as closest geographically to the input geographic location of the patient.

The system in another step, can also operate using software running in electronic memory of the host computer or a networked computer to the operation of generating an appointment for the patient at the location of the chosen eye care professional. The system, using such software, will communicate with the electronic scheduling system of the chosen eye care professional, and ascertain one or a plurality of available appointment dates at appointment times. The available appointment dates at appointment times will be electronically communicated to the patient or parent thereof for input of a chosen appointment. The software of the system provider will then take the step of accessing the scheduling software of the eye care professional and imput the appointment date and time for the patient automatically.

In this fashion, the parent or guardian is given the vision test outcome and an easy and convenient means to contact the local eye care professional for an appointment. If such is handled by electronic communication, then the email or the like may contain an executable link, where the parent or guardian is linked to the appointment scheduling for the local eye care professional so as to make an appointment. In another preferred mode of the system herein, the examination station may be configured to work as a game or the like, and may have prizes awarded or, for example, a “you won” page that is presented to the patient after completing an examination of each eye. Graphics displayed on a video display adjacent to or as part of the examination station would be provided to entice the patient to play the game and, in the course thereof, perform their eyesight examination. The game would direct the patient to employ the lenses on the examination station in the same fashion noted above. The results or outcome and referral to the local eye care professional would also be, as noted above, with the game version.

With respect to the above description, before explaining at least one preferred embodiment of the eyesight examination and professional referral system herein, it is to be understood that the invention is not limited in its application to the details of operation nor the arrangement of the components or the steps set forth in the following description or illustrations in the drawings. The various methods of implementation and operation of the method and system herein are capable of other embodiments and of being practiced and carried out in various ways which will be obvious to those skilled in the art once they review this disclosure. Also, it is to be understood that the phraseology and terminology employed herein are for the purpose of description and should not be regarded as limiting.

Therefore, those skilled in the art will appreciate that the conception upon which this disclosure is based, may readily be utilized as a basis for designing of methods and systems for carrying out eyesight exams and referrals and the several purposes of the present method. Therefore, the objects and claims herein should be regarded as including such equivalent construction, steps, and methodology insofar as they do not depart from the spirit and scope of the present invention.

It is an object of this invention to provide a system to more accurately diagnose the vision of patients, such as children.

It is an additional object of the system herein to provide such a system using positioned eyesight examinations which are fun and interesting to younger patients to increase their attention during vision tests to enhance the outcome.

Yet another object of this invention is the provision of a professional quality eye examination performed remotely on placed examination stations by allowing the user to discern the clearest images for each eye and identify such with an icon or the like which is cross referenced in by software adapted to the task with a database with the appropriate vision correction required.

It is a further object of the invention to provide a concurrent referral and appointment date to adults or children, having identified eyesight issues, to visit a vision professional, proximate to the location of the home or school of the child, who is qualified to treat the identified vision issue.

These together with other objects and advantages which become subsequently apparent reside in the details of the construction and operation of the system herein as more fully hereinafter described and claimed, reference being had to the accompanying drawings forming a part thereof, wherein like numerals refer to like parts throughout.

Further objectives of this invention will be ascertained by those skilled in the art as brought out in the following part of the specification wherein detailed description is for the purpose of fully disclosing the invention without placing limitations thereon.

### BRIEF DESCRIPTION OF DRAWING FIGURE

FIG. 1 shows an example of a wheel located at examination stations for the system herein, having a plurality of examination lenses through which vision examination images are viewed and shows the patient-chosen vision determination image positioned adjacent each examination lens.

FIG. 2 shows an example of viewing of the vision examination images as viewed through the examination station lenses, whereby the user may ascertain and choose the vision determination image adjacent the examination lens they have determined as having the most focused viewing.

FIG. 3 shows an overview of a preferred mode of the system herein where the same examination image, such as an eye chart, is viewable through all of the lenses, so the user can determine which of the lenses provides the clearest examination image when viewed therethrough.

FIG. 4 is an example of the inclusion of a game like operation of the examination station of the system herein which is configured to pique the interest of the patient using a spinning jackpot wheel.

FIG. 5 is a general flow chart of the system herein for performing eye examinations and communicating the outcome thereof in combination with contact or appointment information for a local eye care professional to work with the patient for vision correction if needed.

### DETAILED DESCRIPTION OF THE PREFERRED EMBODIMENTS OF THE INVENTION

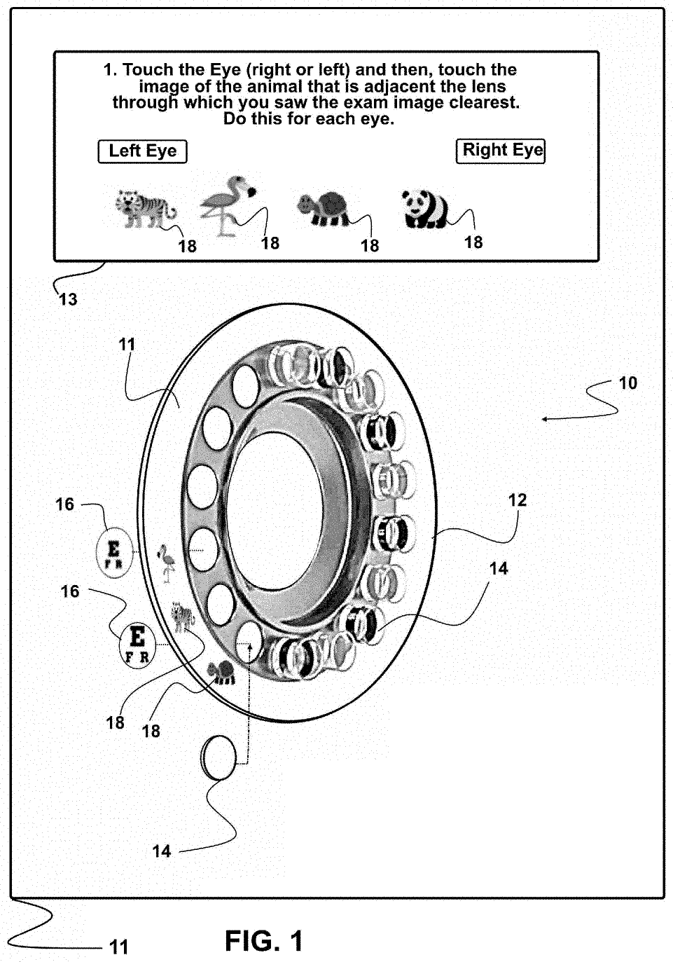

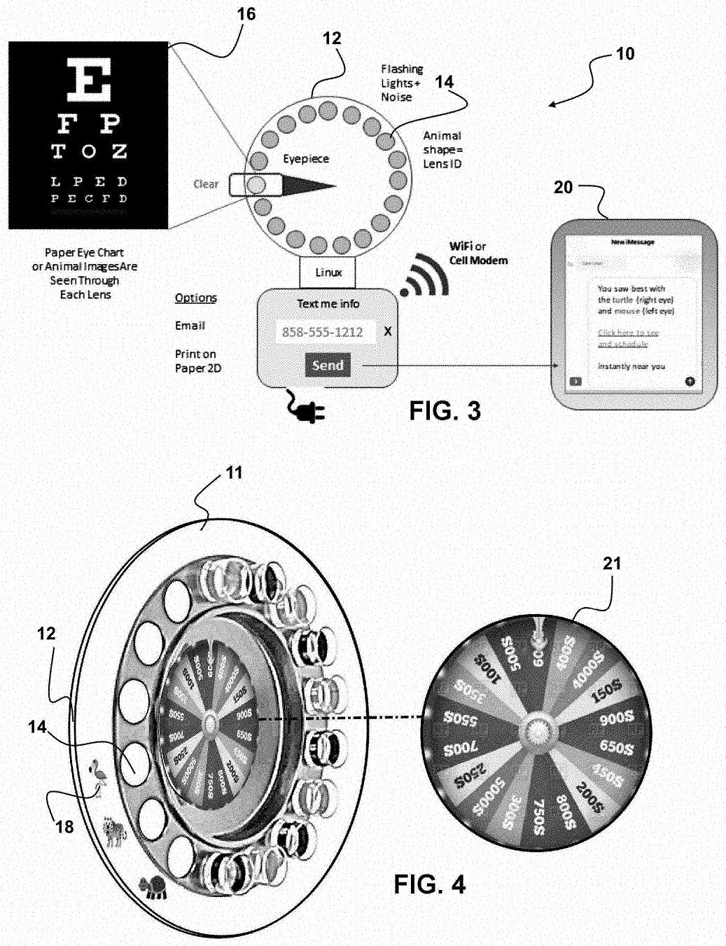

Referring now to the method and system herein shown in simple format by the depictions of FIGS. 1-5. As shown in FIG. 1, an examination station 11 of the system 10 will be physically positioned at a venue where it can be used by patients. The examination station 11, will employ a testing wheel 12 for self examination of the vision of a user or patient. Such will allow for the provision of professional quality eye examinations at remote locations by allowing the user to discern the clearest vision of an exam image 16 for each eye. The examination image 16 is positioned behind each of the plurality of different lenses 14 at the examination station 11. The user will look through each respective lens 14 at the examination image 16 to ascertain through which respective lens 14 the examination image 16 is clearest.

Each respective lens 14 is associated with or identified by a vision determination image 18. This vision determination image 18 is employable, subsequently, for the user to identify and communicate electronically for the system to determine in which respective lens 14 the user saw the clearest version of the examination image 16. In the system herein, the communicated vision determination image 18, for each respective eye of the user, can be cross referenced using software operating in electronic memory to the task of comparing the user chosen determination image 18 with a database of lens or optical vision corrections required and correlated to best correct the vision of each eye of the user based on the chosen determination image 18.

As noted above, once positioned at an eyesight examination station 11, if patient information has not already been input such as at a school, or the patient is an adult and using it at a venue such as a drug store, the patient may be presented with a graphic display 13 having input areas thereon and, if needed, a graphic depiction of typewriter keys, which allow the patient to at least input their name and electronic or mail contact information and a geographic location for the home or work location of the patient. While this input graphic interface is not shown on the display 13 of FIG. 1, such is well known.

This patient information is communicated to a computer running software configured to the task of accepting such patient information and storing it in electronic memory where a patient identifier is assigned, and the location and other input personal information is associated with a patient identifier for the patient in a database file for that patient.

As shown in FIGS. 1-3, the testing wheel 12 is employed by the patient for self testing. The testing wheel 12 has a plurality of examination lenses 14 positioned thereon. Each of the examination lenses 14 are configured to operate optically in a fashion similar to lenses used in a phoropter device employed by vision professionals such as opticians and ophthalmologists. Each of the examination lenses 14 are aligned and associated with a respective vision exam image 16 which is viewed therethrough such as an eye chart or another image.

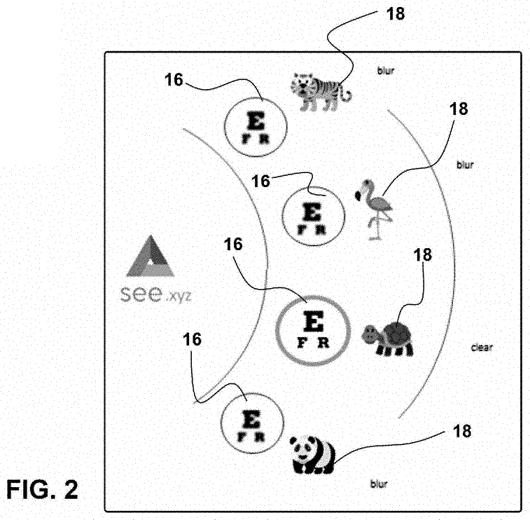

A patient viewing the vision exam image 16, through each of the various examination lenses 14, will find one such viewed vision exam image 16 to be more focused or clearer than the others during the test. Such can be seen in FIG. 2, wherein the vision exam image 16 seen through an aligned examination lens 14 is clearer and is positioned adjacent a vision determination image 18 shown as a depiction of a turtle.

As noted above, the patient will be given an input to choose the vision determination image 18 for each eye which they deem the clearest or most focused such as is shown on the video display 13 in FIG. 1. This choice of the vision determination images 18 is communicated electronically as a vision test outcome 20 to a computing device having a computer process operatively engaged to memory such as network-based non-transitory storage. Running on the system computer in the electronic memory will be vision determination software operating to the task of receiving each vision determination image 18 associated with each respective eye of the user, and generating the vision test outcome 20 for that respective eye. The vision determination software will then associate each such vision test outcome 20 for each eye of the patient identifier assigned to that patient and will store the vision test outcome in electronic memory.

Using this vision test outcome 20, the system using correction determination software, operating in electronic memory of the system computer or server, will operate to the task of ascertaining available optical vision correction, and whether optical correction is needed to bring the vision of the patient to a normal level of vision, such as close to 20/20 vision as optical lenses can achieve.

The available vision correction results of the testing may be communicated to the patient, if they are an adult, or to the parents, guardian, or school of a patient who is a minor. If the available vision correction outcome determines that optical correction is available, as determined by the system for the examined patient, the system 10, using the patient geographic location input by the patient and associated in electronic memory with the patient identifier, will then employ professional location software running in electronic memory to the task of ascertaining the name and location of a local eye care professional office, which is located close or closest by distance to that patient geographic location.

The system, in communications with the patient or parents, will communicate the vison test outcome 20, and in a particularly preferred mode of the system 10, will communicate an executable link and/or contact information for the eye care professional determined close to the patient geographic location of the patient. Such will allow for easy communication to schedule an eye exam appointment followup for the patient and encourage prompt care for the patient, which, as noted, is preferred when the patient is a child and poor vision can generate many problems.

Optionally, instead of providing the executable link to the local eye professional, the system 10, in another step, can also operate using scheduling software running in electronic memory of the host computer or a networked computer, to the operation of generating an appointment for the patent at the location of the determined eye care professional closest to the patient geographic location. The system, using such scheduling software, will electronically communicate with the electronic scheduling system of the chosen eye care professional, and ascertain one or a plurality of available appointment dates at appointment times. The available appointment dates at appointment times will be electronically communicated to the patient or parent thereof for input of a chosen appointment. The scheduling software of the system provider will then take the step of accessing the scheduling software of the eye care professional, and impute the appointment date and time for the patient automatically.

Particularly preferred, in one mode of the system 10, is the inclusion of a game 21 operation for the examination station 11. Such would employ a computer graphic display 13 with graphics to operate the game 21 and, more particularly, gain the ongoing interest of the patient during the test.

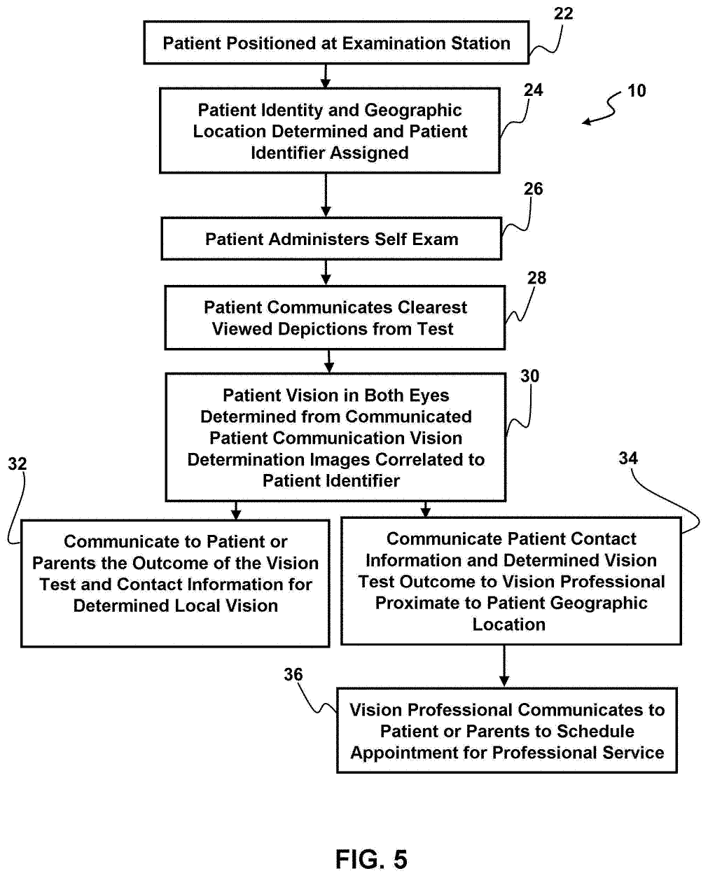

As shown in FIG. 5, the steps of the system would have the patient being positioned 22 at the exam station 11. If not pre-entered, the patient is provided with inputs such as a graphically depicted typewriter on the graphic display 13 for input of patient identity along with a geographic location, such as home address, to associate with the patent and assign the patient identifier 24. Thereafter the eye exam is accomplished 26, as noted above, by the patient viewing the examination image 16 through each of the respective lenses 14.

Subsequent to the exam of each eye of the patient, the patient will choose the vision determination image 18 adjacent the exam lens 14 which yielded the clearest vision 28. The results of the vision determination image 18 for each eye, is communicated to the system provider over a network such as the internet. A graphic interface, such as in FIG. 1, is provided for such input as to the right and left eye. The patient vision test outcome for each of both eyes is determined and associated with the patient identifier 20.

Thereafter, the vision test outcome can be communicated to the patient or parents or guardian of the patient along with contact information for the local vision professional 32. Concurrently and preferred, to make it easy to schedule an appointment for the patient, contact information for the patient may be communicated to the chosen local vision professional 34. This allows, in a preferred step, for the vision professional to contact the patient or parent or guardian to more easily schedule an appointment 36.

The system 10 can encourage widespread use and deployment of stations 11 to the maximum number of schools and the students at such schools, by charging the referred vision professionals a fee for each such referral. Such will enable the system provider to maximize the number of sites where the examination station 11 is deployed, and thereby maximize the number of students who are examined to provide early vision correction to them.

While all of the fundamental characteristics and features of the system for the patient eyesight exam and referral system have been shown and described herein, with reference to particular embodiments thereof, a latitude of modification, various changes and substitutions are intended in the foregoing disclosure and it will be apparent that in some instances, some features of the invention may be employed without a corresponding use of other features without departing from the scope of the invention as set forth. It should also be understood that various substitutions, modifications, and variations may be made by those skilled in the art without departing from the spirit or scope of the invention. Consequently, all such modifications and variations and substitutions are included within the scope of the invention as defined by the following claims.

What is claimed is:

1. A patient eyesight exam and referral system as shown and described herein.
